(19) United States
(12) Patent Application Publication
(10) Pub. No.: US 2026/0049265 A1
(43) Pub. Date: Feb. 19, 2026

(54) SUBSTRATES HAVING WATER-SOLUBLE POLYMER ZONES COMPRISING A FRESHENING ACTIVE AND CONSUMER PRODUCTS COMPRISING THE SAME

(71) Applicant: The Procter & Gamble Company, Cincinnati, OH (US)

(72) Inventors: Rajesh Kant MISHRA, Mason, OH (US); Emily Charlotte BOSWELL, Cincinnati, OH (US); Shaun Thomas BROERING, Ft. Thomas, KY (US); Jessica Mary GREER, Chicago, IL (US); Jack Alan HUNTER, Springboro, OH (US); Jeffrey Scott STIGLIC, New Lenox, IL (US); Elizabeth Anne WILDER, West Chester, OH (US)

(21) Appl. No.: 19/370,887

(22) Filed: Oct. 28, 2025

Related U.S. Application Data
(63) Continuation of application No. 17/071,069, filed on Oct. 15, 2020.
(60) Provisional application No. 62/930,587, filed on Nov. 5, 2019.

(51) Int. Cl. C11D 17/04; A61L 9/04; A61L 15/28; A61L 15/46; B01D 46/00; C11D 1/66; C11D 3/22; C11D 3/37; C11D 3/395; C11D 3/48; C11D 3/50

(52) U.S. Cl. CPC C11D 17/042; A61L 9/042; A61L 15/28; A61L 15/46; B01D 46/0028; B01D 46/0038; C11D 1/662; C11D 3/225; C11D 3/3707; C11D 3/3951; C11D 3/48; C11D 3/505; C11D 17/049; A61L 2209/14

(57) ABSTRACT

A substrate and consumer products made therefrom are provided. The substrate includes a surface and a water-soluble polymer zone disposed on and in contact with the surface. The water-soluble polymer zone includes a freshening active, a water-soluble, film-forming polymer, and a surfactant.

### FIELD

The present invention is directed to substrates having freshening actives, methods of making the same, and consumer products comprising the substrates.

### BACKGROUND

Substrates such as films, non-wovens, foams, are a common component in various comme consumer products. For example, grocery bags, trash bags, sacks, and packaging materials are products that are commonly made from thermoplastic films. Additionally, feminine hygiene products, baby diapers, adult incontinence products, and many other products include thermoplastic films and/or non-wovens to one extent or another. Cleaning implements may include foam and/or non-woven substrates. Consumer products such as trash bags, diapers, and cleaning implements may include freshening actives such as fragrances. Freshening actives that can be volatilized at ambient temperatures to control malodor may be substantially volatilized during the production process, which may involve high temperature melt extrusion of polyethylene or other plastics. This volatilization during the production process can waste valuable volatile fragrance and freshening actives, and change the notes of the fragrance and freshening actives as the components are differentially volatilized. Further, the fragrance and freshening actives that can be applied to the substrate may be limited to materials that will stay attached with the substrate so as not to flake or fall off. As such, it would be beneficial to provide a substrate for a consumer product having freshening actives that are stable until a time when the release of the freshening active is needed or desired.

### SUMMARY

### “Combinations:”

A. A consumer product comprising:
a substrate comprising a surface;
a water-soluble polymer zone disposed on and in contact with the surface, the water-soluble polymer zone comprising a freshening active, a polymer, and a surfactant,
wherein the consumer product is free of an adhesive that is disposed between the surface of the substrate and the water-soluble polymer zone.
   B. The consumer product of Paragraph A, wherein the freshening active is selected from the group consisting of: a perfume raw material, malodor counteractant, intercalated bleach, cleaning active, antibacterial active, and combinations thereof.
   C. The consumer product of Paragraph A or Paragraph B, wherein the freshening active is encapsulated.
   D. The consumer product of any of Paragraphs A through C, wherein the substrate is selected from the group consisting of: a non-woven, a polymeric film, a foam, and combinations thereof.
   E. The consumer product of any of Paragraphs A through D, wherein the polymer is selected from the group consisting of: methyl cellulose, hydroxyethyl methyl cellulose, hydroxypropyl methyl cellulose, hydroxypropyl ethyl cellulose, ethyl cellulose, hydroxyethyl methyl cellulose, and combinations thereof.
   F. The consumer product of any of Paragraphs A through E, wherein the surfactant is selected from the group consisting of: ethoxylated sorbitan, sorbitan, and combinations thereof.
   G. The consumer product of any of Paragraphs A through F, wherein the water-soluble polymer zone comprises at least 70 wt. % of a freshening active.
   H. The thermoplastic film of any of Paragraphs A through G further comprising a plurality of discontinuously dispersed water-soluble polymer zones.
   I. The consumer product comprising of any of Paragraphs A through H, wherein the consumer product is selected from the group consisting of: wipe, duster, dry mop pad, air filter, dryer sheet, bag, film, diaper, sanitary napkins, incontinence products, fiber-based detergent, paper towel, toilet paper, foam sponge, and combinations thereof.
   J. A method of making a consumer product, the method comprising the steps of:
providing a substrate;
depositing a liquid, water-soluble polymer composition onto one or more locations of the substrate, wherein the liquid, water-soluble polymer composition comprising a freshening active, a polymer, and a surfactant; and
drying the liquid, water-soluble polymer composition on the surface of the substrate to form one or more water-soluble polymer zones.
   K. The method of Paragraph J, wherein the freshening active is selected from the group consisting of: a perfume raw material, malodor counteractant, an intercalated bleach, cleaning active, antibacterial active, and combinations thereof.
   L. The method of Paragraph J or Paragraph K, wherein the freshening active is encapsulated.
   M. The method of any of Paragraphs J through L, wherein the substrate is selected from the group consisting of: a non-woven, a polymeric film, a foam, and combinations thereof.
   N. The method of any of Paragraphs J through M wherein the polymer is selected from the group consisting of: methyl cellulose, hydroxyethyl methyl cellulose, hydroxypropyl methyl cellulose, hydroxypropyl ethyl cellulose, ethyl cellulose, hydroxyethyl methyl cellulose, and combinations thereof.
   O. The method of any of Paragraphs J through N, wherein the surfactant is selected from the group consisting of: ethoxylated sorbitan, sorbitan, and combinations thereof.
   P. The method of any of Paragraphs J through O, wherein the water-soluble polymer zone comprises at least 70 wt. % of a freshening active.
   Q. The method of any of Paragraphs J through P, wherein the step of depositing a liquid, water-soluble polymer composition onto one or more locations of the substrate further comprises depositing the liquid, water-soluble polymer composition onto multiple locations of the substrate, and wherein the step of drying the liquid, water-soluble polymer composition on the surface of the substrate to form one or more water-soluble polymer zones further comprises drying the liquid, water-soluble polymer composition on the surface of the substrate to form multiple, discontinuously dispersed water-soluble polymer zones.
   R. The method of any of Paragraphs J through Q, wherein the consumer product is selected from the group consisting of: wipe, duster, dry mop pad, air filter, dryer sheet, bag, film, diaper, sanitary napkins, incontinence products, fiber-based detergent, paper towel, toilet paper, foam sponge, and combinations thereof.
   S. The method of any of Paragraphs J through R, wherein the water-soluble polymer composition has a contact angle less than 70 degrees on the thermoplastic film.

### BRIEF DESCRIPTION OF THE DRAWINGS

FIG. 1 is a schematic of a plan view of film comprising a plurality of water-soluble polymer zones.

FIG. 2 is a perspective view of an exemplary, non-limiting example of a thermoplastic bag.

FIG. 3 is a schematic, sectional view of a thermoplastic bag comprising water-soluble polymer zones.

FIG. 4 is a plan view of an exterior sidewall of a thermoplastic bag, illustrating freshening active placement regions of the thermoplastic bag.

FIG. 5 illustrates a manufacturing process for making thermoplastic films having water-soluble polymer zones disposed thereon and then producing plastic bags therefrom.

FIG. 6 is a plot of the modulus of the water-soluble polymer zones at varying relative humidity.

FIG. 7 is a plot of the fragrance and malodor intensity of exemplary and comparative water-soluble polymer compositions.

FIG. 8 is a plot of time to bloom for a fragrance and a fragrance intensity of exemplary water-soluble polymer zones comprising an encapsulated freshening active disposed between layers of a multi-layer thermoplastic bag compared with water-soluble polymer zones disposed on the inside of a thermoplastic bag.

FIG. 9 is a plot of the malodor reduction of a water-soluble polymer zones comprising an intercalated bleach active disposed between layers of a multi-layer thermoplastic bag compared with water-soluble polymer zones disposed on the inside of a thermoplastic bag.

### DETAILED DESCRIPTION

The present invention may be understood more readily by reference to the following detailed description of illustrative and preferred compositions. It is to be understood that the scope of the claims is not limited to the specific products, methods, conditions, or parameters described herein, and that the terminology used herein is not intended to be limiting of the claimed invention.

All percentages and ratios used herein are by weight of the total composition. All numeric ranges are inclusive of narrower ranges; delineated upper and lower range limits are interchangeable to create further ranges not explicitly delineated. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity.

The following definitions may be useful in understanding the present disclosure.

“Molecular weight” means the weight average molecular weight unless otherwise stated. Molecular weight is measured using industry standard method, gel permeation chromatography (“GPC”).

“Nonwoven” means a porous, fibrous material made from continuous (long) filaments (fibers) and/or discontinuous (short) filaments (fibers) by processes such as, for example, spun bonding, melt blowing, carding, and the like. Nonwoven webs do not have a woven or knitted filament pattern.

As used herein, the terms “freshening active” refer to a composition that effects (e.g., changes and/or masks) odors in at least one manner. For example, the “freshening active” may absorb malodorants (e.g., foul smell odors) and/or may release fragrance materials. Furthermore, the “freshening active” may mask (e.g., cover up) and/or neutralize malodorants. As used herein the term “neutralize” or any of its derivative terms refers to an ability of a compound or product to reduce or eliminate malodorous compounds. Odor neutralization may be partial, affecting only some of the malodorous compounds in a given context, or affecting only a portion of a malodorous compound. A malodorous compound may be neutralized by chemical reaction resulting in a new chemical entity, by sequestration, by chelation, by association, or by any other interaction rendering the malodorous compound less malodourous or non-malodorous.

As used herein, the term “odor” refers to any substance that can stimulate an olfactory response in a human; i.e., sense of smell.

As used herein the term “malodor” and any of its derivative terms refers to an odor that is generally considered unpleasant, obnoxious, or nauseating by the general population, such as the broad spectrum of odors associated with household trash, including odors related to stale urine, feces, vomitus, and putrefying organic materials, e.g., food waste, in common household trash, such as garlic, onions, fish, etc.

As used herein, the term “substantially,” in reference to a given parameter, property, or condition, means to a degree that one of ordinary skill in the art would understand that the given parameter, property, or condition is met within a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, any relational terms such as “first,” “second,” and “third,” “inner,” “outer,” “upper,” “lower,” “side,” “top,” “bottom,” etc. are for clarity and convenience in understanding the present disclosure and accompanying drawings and does not connote or depend on any specific preference, orientation, or order, except where the context clearly indicates otherwise. For example, the relational terms may refer an orientation of a bag while disposed within a receptacle (e.g., a trash can) for use.

One or more implementations of the present disclosure include a substrate having a freshening active. The substrate may be incorporated into a consumer product.

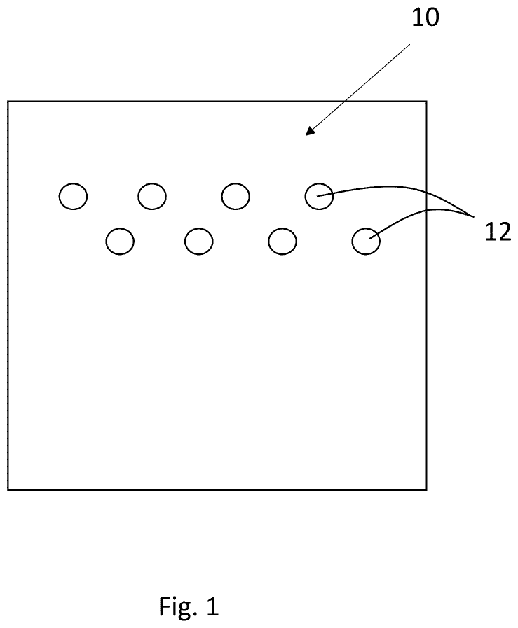

With reference to FIG. 1, the substrate may include a water-soluble polymer zone 12 disposed on and in direct contact with the substrate 10. The water-soluble zone comprises a water-soluble polymer that fix freshening actives to the substrate. The water-soluble polymer may act to adhere the freshening active to the substrate and control the release of the freshening active therefrom. Release of the freshening active may occur through friction, dissolution of the water-soluble polymer, heat, pH change, etc.

The water-soluble polymer composition can comprise one or more freshening actives, such as volatile fragrance materials, desiccant materials, antimicrobial agents, deodorizing agents, stabilized oxidants, functional nanoparticles, and the like. As a result, the freshening active reduces an amount of malodorant molecules that permeate from or through the film, masks malodorant molecules, and/or otherwise neutralizes or blocks malodor.

A freshening active may at least partially absorb and/or trap malodorant molecules. In other words, the freshening active can “catch” the malodorant molecules. By absorbing and/or trapping the malodorant molecules, the freshening active can help reduce or prevent the malodorant molecules from permeating from or through the film.

Since the water-soluble polymer fixes the freshening active on the substrate, the freshening actives can comprise substances not typically used for “scenting” films. For example, the water-soluble polymer composition can prevent the freshening active from prematurely releasing from the substrate, potentially coming into contact with a consumer, or causing premature activation of the freshening active. As a result, the film may employ liquids, sticky, oily, and/or solid materials as a freshening active. The water-soluble polymer composition may be able to control the release or exposure of the freshening actives, allowing for the use of freshening actives that would otherwise be avoided on substrates because the freshening active may be considered a skin irritant or may be associated with inhalation concerns under some use conditions. The use of the water-soluble polymer zone to fix the freshening active to the substrate can also reduce the waste of expensive freshening actives that are not properly adhered to the substrate, that would otherwise not be secured and could be lost in manufacturing or handling before use.

A substrate comprising the water-soluble polymer zones of the present invention may be tailored to provide an enhanced release of one or more freshening actives. Specifically, holding the freshening active in the water-soluble polymer composition may provide control of a rate at which the one or more substances of the freshening active are released and/or a direction in which the one or more substances of the freshening active are released. The substrate may include one or multiple different freshening actives in regions or islands of water-soluble polymer composition disposed on the substrate that release at different times or have different functions/effects. Specific regions of the substrate could be made to release freshening active at different times by using different water-soluble polymer composition formulations with different dissolution times than other water-soluble polymer compositions, and/or different molecular weight polymers that could affect the dissolution times and/or different water-soluble polymer zones having different thicknesses.

The present disclosure includes consumer products made from or with such substrate. For example, such products include, but are not limited to, grocery bags, trash bags, sacks, and packaging materials, feminine hygiene products, baby diapers, adult incontinence products, wipes, dusters, mop pads, air filters, dryer sheets, fiber-based detergents, paper towels, toilet papers, foam sponges, and combinations thereof. Consumer products can also include the same or similar products that are used in a commercial or industrial setting. For ease in description, the figures and bulk of the following disclosure focuses on films and bags. One will appreciate that teachings and disclosure equally applies to other consumer products. For example, nonwovens, woven materials, and/or foams may be used in place of the films described herein.

The water-soluble polymer composition may be disposed at various locations on the substrate. Some substrates may include water-soluble polymer composition with freshening actives at one or more locations that include the same or different freshening actives.

The water-soluble polymer zone is in direct contact with the surface of the substrate, without the need for a supplemental adhesive to join the water-soluble polymer zone and the surface of the substrate. That is, the substrate may be free of an adhesive that is disposed between and/or adheres the water-soluble polymer zone with the surface of the substrate. For example, the substrate may be free of a pressure-sensitive adhesive that is disposed between and/or adheres the water-soluble polymer zone with the surface of the film As used herein, an “adhesive” includes acrylic and methacrylic ester homo- or copolymers, butyl rubber-based systems, silicones, urethanes, vinyl esters and amides, olefin copolymer materials, natural or synthetic rubbers, hot-melt adhesives; polyethylenes; polysiloxanes; polyisobutylenes; polyacrylates; polyacrylamides; polyurethanes; plasticized ethylene vinyl acetate copolymers; and tacky rubbers such as polyisobutene, polybutadiene, polystyreneisoprene copolymers, polystyrene-butadiene copolymers, and neoprene (polychloroprene) and combinations thereof. Adhesives may be present on other portions of the substrate or consumer product so long as the adhesive is not used to join the water-soluble polymer zone with the substrate. As used herein, the “adhesive” is considered a different component from the water-soluble film forming polymer and the freshening active.

### Substrate

Suitable substrates include woven webs, nonwoven webs or films, such as polymeric web materials, (apertured formed) thermoplastic films, (apertured) plastic films, hydroformed thermoplastic films, reticulated thermoplastic films and combinations, e.g., laminates thereof. The substrates may be porous foams, reticulated foams, and thermoplastic scrims; paper tissue or combinations thereof.

Exemplary films may include thermoplastic polyolefins, including polyethylene and copolymers thereof and polypropylene and copolymers thereof. The olefin-based polymers may include ethylene or propylene-based polymers such as polyethylene, polypropylene, and copolymers such as ethylene vinyl acetate (EVA), ethylene methyl acrylate (EMA) and ethylene acrylic acid (EAA), or blends of such polyolefins.

Other examples of polymers suitable for use as films in accordance with the present invention may include elastomeric polymers. Suitable elastomeric polymers may also be biodegradable or environmentally degradable. Suitable elastomeric polymers may include recycled elastomeric polymers either alone or in combination with virgin elastomeric polymers. Suitable elastomeric polymers for the film include poly(ethylene-butene), poly(ethylene-hexene), poly(ethylene-octene), poly(ethylene-propylene), poly(styrene-butadiene-styrene), poly(styrene-isoprene-styrene), poly(styrene-ethylene-butylene-styrene), poly(ester-ether), poly(ether-amide), poly(ethylene-vinylacetate), poly(ethylene-methylacrylate), poly(ethylene-acrylic acid), oriented poly(ethylene-terephthalate), poly(ethylene-butylacrylate), polyurethane, poly(ethylene-propylene-diene), ethylene-propylene rubber, nylon, etc. Suitable biodegradable elastomeric polymers include starch, polylactides, polyhydroxyalkanoates, bio-identical renewable resins such as bio-polyethylene, and the like.

Some of the examples and description herein below refer to films formed from linear low-density polyethylene. The term “linear low density polyethylene” (LLDPE) as used herein is defined to mean a copolymer of ethylene and a minor amount of an olefin containing 4 to 10 carbon atoms, having a density of from about 0.910 to about 0.926, and a melt index (MI) of from about 0.5 to about 10. For example, some examples herein use an octene comonomer, solution phase LLDPE (MI=1.1; ρ=0.920). Additionally, other examples use a gas phase LLDPE, which is a hexene gas phase LLDPE formulated with slip/AB (MI=1.0; ρ=0.920). Still further examples use a gas phase LLDPE, which is a hexene gas phase LLDPE formulated with slip/AB (MI=1.0; ρ=0.926). One will appreciate that the present disclosure is not limited to LLDPE, and can include “high density polyethylene” (HDPE), “low density polyethylene” (LDPE), and “very low density polyethylene” (VLDPE). Indeed, films made from any of the previously mentioned thermoplastic materials or combinations thereof can be suitable for use with the present disclosure.

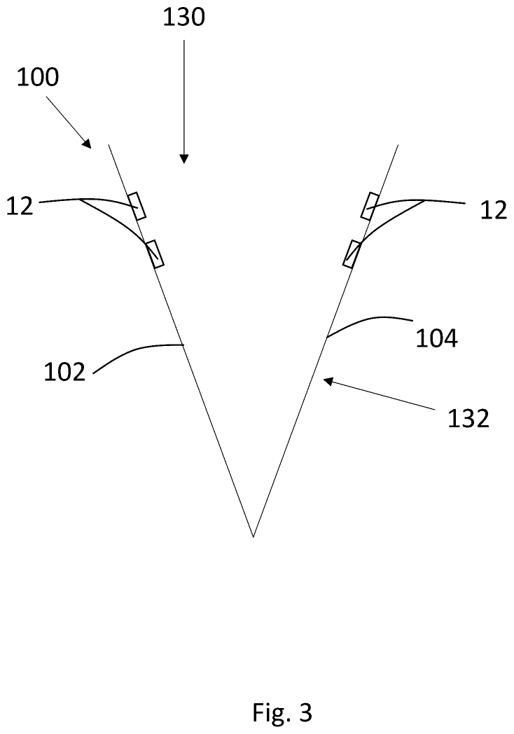

Some films of the present invention may include any flexible or pliable thermoplastic material that may be formed or drawn into a web or film. Furthermore, the thermoplastic materials may include a single layer or multiple layers as described in further detail below in regard to FIGS. 3A-3C. The thermoplastic material may be opaque, transparent, translucent, or tinted. Furthermore, the thermoplastic material may be gas permeable or impermeable.

As used herein, the term “flexible” refers to materials that are capable of being flexed or bent, especially repeatedly, such that they are pliant and yieldable in response to externally applied forces. Accordingly, “flexible” is substantially opposite in meaning to the terms inflexible, rigid, or unyielding. Materials and structures that are flexible, therefore, may be altered in shape and structure to accommodate external forces and to conform to the shape of objects brought into contact with them without losing their integrity. In accordance with further prior art materials, web materials are provided which exhibit an “elastic-like” behavior in the direction of applied strain without the use of added traditional elastic. As used herein, the term “elastic-like” describes the behavior of web materials which when subjected to an applied strain, the web materials extend in the direction of applied strain, and when the applied strain is released the web materials return, to a degree, to their pre-strained condition.

The film may include slip agents, anti-block agents, voiding agents, or tackifiers. The film may be devoid of voiding agents. Some examples of inorganic voiding agents, which may further provide odor control, include the following but are not limited to: calcium carbonate, magnesium carbonate, barium carbonate, calcium sulfate, magnesium sulfate, barium sulfate, calcium oxide, magnesium oxide, titanium oxide, zinc oxide, aluminum hydroxide, magnesium hydroxide, talc, clay, silica, alumina, mica, glass powder, starch, charcoal, zeolites, any combination thereof, etc. Organic voiding agents, polymers that are immiscible in the major polymer matrix, can also be used. For instance, polystyrene can be used as a voiding agent in polyethylene and polypropylene films.

The film may include natural oils. For example, the additives may include thyme oil, mint oil, lemon grass oil, tea tree oil, cinnamon bark oil, methyl jasmonate, etc. Yet further additives may include zinc pyrithione (“ZPT”) and copper pyrithione (“CPT”), which inhibit microbial growth.

One of ordinary skill in the art will appreciate that manufacturers may form the films or webs to be used with the present disclosure using a wide variety of techniques. For example, a manufacturer can form precursor mix of the thermoplastic material and one or more additives. The manufacturer can then form the film(s) from the precursor mix using conventional flat or cast extrusion or coextrusion to produce monolayer, bilayer, or multilayer films. Alternatively, a manufacturer can form the films using suitable processes, such as, a blown film process to produce monolayer, bilayer, or multilayer films. If desired for a given end use, the manufacturer can orient the films by trapped bubble, tenterframe, or other suitable process. Additionally, the manufacturer can optionally anneal the films thereafter.

The film-making process may include a procedure known as “orientation.” The orientation of a polymer is a reference to its molecular organization, i.e., the orientation of molecules relative to each other. Similarly, the process of orientation is the process by which directionality (orientation) is imposed upon the polymeric arrangements in the film. The process of orientation is employed to impart desirable properties to films, including making cast films tougher (higher tensile properties). Depending on whether the film is made by casting as a flat film or by blowing as a tubular film, the orientation process can require different procedures. This is related to the different physical characteristics possessed by films made by the two conventional film-making processes; casting and blowing. Generally, blown films tend to have greater stiffness and toughness. By contrast, cast films usually have the advantages of greater film clarity and uniformity of thickness and flatness, generally permitting use of a wider range of polymers and producing a higher quality film.

When a film has been stretched in a single direction (monoaxial orientation), the resulting film can exhibit strength and stiffness along the direction of stretch, but can be weak in the other direction, i.e., across the stretch, often splitting when flexed or pulled. To overcome this limitation, two-way or biaxial orientation can be employed to more evenly distribute the strength qualities of the film in two directions. Most biaxial orientation processes use apparatus that stretches the film sequentially, first in one direction and then in the other.

In one or more implementations, the films may be blown film, or cast film. Blown film and cast film is formed by extrusion. The extruder used can be a conventional one using a die, which will provide the desired gauge. Some useful extruders are described in U.S. Pat. Nos. 4,814,135; 4,857,600; 5,076,988; 5,153,382; each of which are incorporated herein by reference in their entirety. Examples of various extruders, which can be used in producing the films to be used with the present disclosure, can be a single screw type modified with a blown film die, an air ring, and continuous take off equipment.

A manufacturer can use multiple extruders to supply different melt streams, which a feed block can order into different channels of a multi-channel die. The multiple extruders can allow a manufacturer to form a film with layers having different compositions. Such film may later be non-continuously laminated with another layer of film to provide the benefits of the present disclosure.

In a blown film process, the die can be an upright cylinder with a circular opening. Rollers can pull molten plastic upward away from the die. An air-ring can cool the film as the film travels upwards. An air outlet can force compressed air into the center of the extruded circular profile, creating a bubble. The air can expand the extruded circular cross section by a multiple of the die diameter. This ratio is called the “blow-up ratio.” When using a blown film process, the manufacturer can collapse the film to double the plies of the film. Alternatively, the manufacturer can cut and fold the film, or cut and leave the film unfolded.

The extrusion process can orient the polymer chains of the blown film. The “orientation” of a polymer is a reference to its molecular organization, i.e., the orientation of molecules or polymer chains relative to each other. In particular, the extrusion process can cause the polymer chains of the blown film to be predominantly oriented in the machine direction. The orientation of the polymer chains can result in an increased strength in the direction of the orientation. As used herein predominately oriented in a particular direction means that the polymer chains are more oriented in the particular direction than another direction. One will appreciate, however, that a film that is predominately oriented in a particular direction can still include polymer chains oriented in directions other than the particular direction. Thus, the initial or starting films (films before being stretched or bonded or laminated in accordance with the principles described herein) can comprise a blown film that is predominately oriented in the machine direction.

The process of blowing up the tubular stock or bubble can further orient the polymer chains of the blown film. In particular, the blow-up process can cause the polymer chains of the blown film to be bi-axially oriented. Despite being bi-axially oriented, the polymer chains of the blown film may be predominantly oriented in the machine direction (i.e., oriented more in the machine direction than the transverse direction).

The films of one or more implementations of the present disclosure can have a starting gauge between about 0.1 mils to about 20 mils, suitably from about 0.2 mils to about 4 mils, suitably in the range of about 0.3 mils to about 2 mils, suitably from about 0.6 mils to about 1.25 mils, suitably from about 0.9 mils to about 1.1 mils, suitably from about 0.3 mils to about 0.7 mils, and suitably from about 0.4 mils and about 0.6 mils. Additionally, the starting gauge of films of one or more implementations of the present disclosure may not be uniform. Thus, the starting gauge of films of one or more implementations of the present disclosure may vary along the length and/or width of the film.

As an initial matter, one or more layers of the films described herein can comprise any flexible or pliable material comprising a thermoplastic material and that can be formed or drawn into a web or film. As described above, the film may include a single layer or a plurality of layers of thermoplastic film. Each individual film layer may itself include a single layer or multiple layers. In other words, the individual layers of the film may each themselves comprise a plurality of laminated layers. Such layers may be significantly more tightly bonded together than the bonding provided by the purposely weak discontinuous bonding in the finished film. Both tight and relatively weak lamination can be accomplished by joining layers by mechanical pressure, joining layers with adhesives, joining with heat and pressure, spread coating, extrusion coating, and combinations thereof. Adjacent sub-layers of an individual layer may be coextruded. Coextrusion results in tight bonding so that the bond strength is greater than the tear resistance of the resulting laminate (i.e., rather than allowing adjacent layers to be peeled apart through breakage of the lamination bonds, the film will tear).

The surface of one or both sides of the film can be embossed before applying the water-soluble polymer composition. Applying the water-soluble polymer composition onto an embossed surface may enhance adherence of the water-soluble polymer composition.

Suitable substrates include web material (e.g., woven or nonwoven web) comprising natural fibers or synthetic fibers or combinations thereof.

Examples of natural fibers may include cellulosic natural fibers, such as fibers from hardwood sources, softwood sources, or other non-wood plants, animal fibers such as wool, silk, fur, and hair.

The synthetic fibers can be any material, such as, but not limited to, those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof.

Further, the synthetic fibers can be a single component (i.e., single synthetic material or mixture makes up entire fiber), bi-component (i.e., the fiber is divided into regions, the regions including two or more different synthetic materials or mixtures thereof and may include co-extruded fibers and core and sheath fibers) and combinations thereof. Bi-component fibers can be used as a component fiber of the web material, and/or they may be present to act as a binder for the other fibers present in the web material.

Any or all of the synthetic fibers may be treated before, during, or after manufacture to change any desired properties of the fibers. For example, the fibers may be coated with the block copolymer described herein instead of or in addition to applying the block copolymer to the substrate. The terms “coated substrate” or “substrate coated with” as used herein are intended to encompass such embodiments as well.

In some embodiments the substrates herein may be or may comprise a nonwoven web material, whereby said nonwoven web may be manufactured by a wide number of known techniques. Nonlimiting examples of techniques include spunbonding, carding, wet-laid, air-laid, meltblown, needle-punching, mechanical entangling, thermo-mechanical entangling, hydroentangling, calender bonding and combination thereof.

The substrate may be or may comprise a laminate web of two or more nonwoven webs. The laminate web may comprise spunbond layer(s) (S), and/or meltblown layer(s) (M), and/or carded layer(s). Suitable laminate webs include, but are not limited to, SS, SSS, SMS, SMMS or SMMMS. In some embodiments, the laminate web may comprise nanofibers having a diameter of less than 1 μm.

The substrate may further comprise laminates of nonwoven layers with film layers.

The substrate may comprise or consist of a foam material. Suitable foams for use herein are selected from the group of foams consisting of: polyurethane foams; polypropylene foams; polyethylene foams; cellulose foam sponges; naturally occurring sponges; open-cell polyester foams; and cross-linked polyethylene foams; and combinations thereof. The foam may comprise a melamine-formaldehyde resin foam. The foam may be heat-compressed. For example, the foam may be a heat-compressed, melamine-formaldehyde resin foam.

### Water-Soluble Polymer Composition

The water-soluble polymer composition, for making a water-soluble polymer zone, comprises a water-soluble, film-forming polymer, a freshening active comprising an amount of a freshening active agent sufficient to combat malodorous components in or around the substrate, and water. The water-soluble polymer composition is applied as a liquid onto the substrate. The majority of the water in the water-soluble polymer composition evaporates, leaving a dry or substantially dry water-soluble polymer zone on the substrate. The water-soluble polymer zone secures the freshening active to the substrate until an activation activity occurs. The activation activity may include friction, moisture, heat, pH change, the like, and combinations thereof. The water-soluble polymer zone dissolves in the presence of moisture from the surrounding environment.

The water-soluble polymer zone, prior to exposure to moisture from the environment, is disposed on the substrate as a dry water-soluble polymer composition. The freshening active may only be minimally released, if at all, from the water-soluble polymer zone in the dry form prior to use and/or prior to exposure to moisture from the environment and/or in the absence of moisture or water. Without being bound by theory, the water-soluble polymer zone or water-soluble polymer composition dissolves, disintegrates, and/or loses its physical integrity when exposed to moisture from the surrounding environment. As the water-soluble polymer zone or water-soluble polymer composition softens and/or dissolves, the freshening active may be released. By securing the freshening active in the water-soluble polymer zone until an activation activity occurs, the freshening active can counteract malodor at a time when the malodor in and around the substrate is approaching a level undesirable to a consumer.

Some water-soluble polymer zones may include freshening actives that are activated by friction. Such water-soluble polymer zones may release the freshening active upon an applied frictional force, with or without the water-soluble polymer zone dissolving or losing its physical integrity.

Prior to release of the freshening active, the water-soluble polymer zone is substantially free of water or may comprise less than about 15%, 12%, or 10%, water, or comprise about 0.001% to about 15% water, or about 0.05% to about 10%, water, by weight of the water-soluble polymer zone, based on the total weight of the water-soluble polymer zone.

Preferred water-soluble, film-forming polymers may be selected from polyethylene oxide polymers, polyvinyl alcohols, polyvinyl pyrrolidone, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatin, natural gums such as xanthan and carrageenan, polyacrylates and water-soluble acrylate copolymers, polymethacrylates, methylcellulose, carboxymethylcellulose, hydroxyethyl cellulose, hydroxypropyl methylcellulose, dextrin, maltodextrin, salts thereof, and combinations thereof.

Additional water-soluble, film-forming polymers for the water-soluble polymer zone may be selected from polyethylene glycol, pullulan, carbohydrate polymers such as natural polysaccharide or derivates including pectin and derivatives, sodium alginate, methyl methacrylate copolymer, carboxyvinyl polymer, amylase, pectin, chitin, chitosan, levan, elsinan, collagen, gelatine, zein, gluten, soy protein isolate, whey protein isolate, casein, gums (such as guar, gum Arabic, tragacanth gum, xanthan gum, gellan sodium salt, gum ghatti, okra gum, karaya gum, locust bean gum, tara gum, quince seed gum, fenugreek seed gum, scleroglucan, psyllium seed gum, tamarind gum, oat gum, quince seed gum, rhizobium gum, biosynthetic gums, Khaya grandifolia gum, pectin, arabian, Konjac mannan, alactomannan, funoran, acetan, welan, rhamsan, furcelleran, succinoglycan, scleroglycan, and dextran, flaxseed gum), propyleneglycol, alginate, starches (such as amylose, amylopectin, modified starches, hydroxyethyl starch, carboxymethyl starch, high amylose starch, hydrooxypropylated high amylose starch, biosynthetic processed starch, starches such as rice, corn, potato, and wheat), dextrans, dextrins and maltodextrins, konjac, acemannan from aloe, carrageenans, scleraglucan, succinoglucan, larch arabinogalactan, chondroitin sulfates, hyaluronic acid, curdlan, deacetylatedkonjac, water-soluble non-gelling polypeptide or protein (such as gelatins, albumins, milk proteins, soy protein, and whey proteins), hydrocolloids (such as synthetic hydrocolloids exemplified by polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrollidone, polyethylene glycols, polyethylene oxides, polyvinyl alcohols, pluronics, tetronics, and other block co-polymers, carboxyvinyl polymers, and colloidal silicon dioxide, soluble polyesters, natural seaweeds, natural seed, natural plant exudates, natural fruit extracts, glycyrrhizic acid, polyacrylic acid, vinyl polymers, cationic polymers, acrylic polymers (such as sodium polyacrylate, polyethyleacrylate and polyacrylamide), and combinations.

The water-soluble, film-forming polymers may be selected from the group consisting of polyethylene oxide polymer, polyvinyl alcohols, polyvinyl alcohol copolymers, starch, methylcellulose, carboxymethylcellulose sodium, hydroxyethyl cellulose, hydroxypropyl methylcellulose, hydroxypropyl cellulose, and combinations. Preferred polymers, copolymers or derivatives thereof suitable for use as water-soluble polymer composition for the water-soluble polymer zone are selected from polyethylene oxides, methylcellulose, hydroxypropyl methylcellulose, hydroxypropyl cellulose, and combinations thereof.

The water-soluble film forming polymers may be made from polyethylene oxides such as polyethylene oxide films or polyethylene glycol, and include POLYOX, sold by the Dow Chemical Company. Polyethylene oxides include POLYOX WSR N-10 (having a molecular weight of 10,000), WSR N-80 (with a molecular weight of about 200,000), WSR N750 (with a molecular weight of about 300,000) of corresponding solubility characteristics. In an aspect, the water-soluble film comprises a polyethylene oxide having a molecular weight from about 500 to about 10,000,000 or from about 10,000 to about 1,000,000 or from about 100,000 to about 300,000 or from about 150,000 to about 250,000.

Other water-soluble, film-forming polymers may comprise plasticized methylcellulose and/or plasticized hydroxypropyl methylcellulose and/or plasticized hydroxypropylcellulose. As used herein, “plasticized” means a composition of methylcellulose or hydroxypropyl methyl cellulose or hydroxypropylcellulose, and plasticizer wherein the plasticizer is used at a level of from about 2% to about 80% or about 2% to about 60%, or from about 10% to about 50% or from about 20% to about 45% by weight of this composition.

Most preferably, the water-soluble, film-forming polymer may be selected from the group consisting of: methyl cellulose, hydroxyethyl methyl cellulose, hydroxypropyl methyl cellulose, hydroxypropyl ethyl cellulose, ethyl cellulose, hydroxyethyl methyl cellulose, and combinations thereof. It has been found that such cellulose materials adhere well to the film and do not flake-off from the surface of the film after the water-soluble polymer composition is dried on the film.

The hydroxypropyl methylcellulose may be METHOCEL™ E5LV, a water-soluble cellulose ether of low viscosity available from Dow/Coloron LTD and having a viscosity is about 4.0-6.0 mPa·s, 2% in water at 20° C. Other METHOCEL™ grades that may be used include METHOCEL™ E3LV (viscosity is about 2.4-3.6 mPa·s, 2% in water at 20° C.), METHOCEL™ E6LV (viscosity is about 4.8-7.2 mPa·s, 2% in water at 20° C.), METHOCEL™ E15LV (viscosity is about 12-18 mPa·s, 2% in water at 20° C.), METHOCEL™ E50LV (viscosity is about 40-60 mPa·s, 2% in water at 20° C.), and METHOCEL™ K3LV (viscosity is about 2.4-3.6 mPa·s, 2% in water at 20° C.).

The polyethylene oxide polymers or cellulose ether may be combined with additional polymers, for example, polymers, copolymers or derivatives thereof which may be other water-soluble film forming polymers. The additional polymers may be selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatin, natural gums such as xanthan and carrageenan, polyacrylates and water-soluble acrylate copolymers, polymethacrylates, methylcellulose, carboxymethylcellulose, hydroxyethyl cellulose, hydroxypropyl methylcellulose, dextrin, maltodextrin, salts thereof, and combinations thereof. In an aspect, the water-soluble polymer zone comprises polyethylene oxide polymer and an additional polymer selected from the group consisting of polyvinyl alcohols, polyvinyl alcohol copolymers, starch, methylcellulose, carboxymethylcellulose sodium, hydroxyethyl cellulose, hydroxypropyl methylcellulose and combinations.

Also suitable are mixtures of polyethylene oxide polymers having different molecular weights. The additional polymers may have weight average molecular weights of about 1,000 to 1,000,000 daltons (Da), (e.g., about 50,000 to 300,000 Da or about 20,000 to 150,000 Da). The level of water-soluble, film-forming polymer in the water-soluble film in the dry state may be about 20% to about 90%, or about 45% to about 85% or about 50% to about 70% by weight of the water-soluble polymer zone.

The water-soluble, film-forming polymers may be a combination of polyethylene oxide such as POLYOX WSR N-80 and methylcellulose or hydroxypropyl methylcellulose such as METHOCEL™ ESLV and optionally a plasticizer. In further aspects, the water-soluble film/fluid may be a combination of polyethylene oxide and ethanol (e.g. such as a 20% solution of polyethylene oxide and the rest a 66:33 ethanol:water mixture).

The water-soluble polymer composition may also comprise one or more plasticizers. For example, it can be beneficial to add plasticizers at a level of from about 2% to about 80% or about 2% to about 60%, by weight of the water-soluble polymer composition or the water-soluble film-forming polymer. The plasticizers may be, for example, glycerol, ethylene glycol, diethylene glycol, hexylene glycol, triethylene glycol, propylene glycol, polyethylene glycol, polypropyl glycol, alkyl citrate, sorbitol, pentaerythritol, glucamine, N-methylglucamine, sodiumcumenesulfonate and mixtures thereof. In one aspect, the plasticizer is glycerol. Other plasticizers may include vegetable oil, polysorbitols, polyethylene oxide, dimethicone, mineral oil, paraffin, C1-C3 alcohols, dimethyl sulfoxide, N, N-dimethylacetamide, sucrose, corn syrup, fructose, dioctyl-sodium-sulfo-succinate, triethyl citrate, tributyl citrate, 1,2-propylenglycol, mono, di- or triacetates of glycerol, natural gums, citrates, and mixtures thereof.

The water-soluble polymer composition may comprise one or more surfactants for solubilizing water-insoluble materials, such as water-insoluble freshening actives. Surfactants that are suitable for use in the aqueous compositions of the present invention can be any of those suitable for use in household cleaning, fabric treatment or deodorizing compositions. These include anionic, nonionic, cationic, ampholytic and zwitterionic detergents.

Examples of anionic surfactants include C8-C22 alkyl sulfates, alkylbenzene sulfonates having from 9 to 15 carbon atoms in the alkyl group, alkyl ethyleneoxide ether sulfates having from 8-22 carbon atoms in the alkyl chain and from 1 to 30 ethylene oxide groups, and Cg to C22 fatty acid soaps. Examples of nonionic surfactants include condensates of from 3 to 30 moles of ethylene oxide with an aliphatic alcohol of 8 to 22 carbon atoms, condensates of 5 to 30 moles of ethylene oxide with an alkyl phenol wherein the alkyl contains 9 to 15 carbon atoms, and Cg to C22 alkyl dimethyl amine oxides. In one embodiment, the nonionic surfactant is a secondary alcohol ethoxylate known as Tergitol™ 15-S, available from The Dow Chemical Company. Other examples of nonionic surfactants include ethoxylated sorbitans, including polysorbate 20, 25, 40, 60, 65, 80 and 120; sorbitans, including: sorbitan stearate and sorbitan X stearate where X is mono, di, tri, iso, diiso, triiso, sesqui, sesquiiso, sorbitan laurate and sorbitan X laurate where X is mono, and other combinations; and combinations thereof. Examples of ampholytic and zwitterionic surfactants are found in U.S. Pat. No. 3,929,678, Laughlin et al, issued Dec. 30, 1975 at Col, 19, line 38 through Col. 22 line 48. Examples of cationic surfactants are tetraalkyl quaternary ammonium salts having at least one alkyl chain of 8 to 22 carbon atoms, wherein the other alkyl groups can contain from 1 to 22 carbon atoms and wherein the anionic counterion is halogen, ethylsulfate or methylsulfate. The term “household cleaning and fabric treatment and deodorizing compositions” herein includes fabric laundering, softening and freshening compositions, and floor, rug and other household surface treatment compositions where it is desired to clean and/or impart a beneficial treatment or property to the surface. Additional surfactants are disclosed in U.S. Pat. No. 3,664,961 to Norris, issued May 23, 1972.

The water-soluble polymer composition can comprise one or more additional ingredients, including bulking agents, fillers, diluents, stabilizing agents, emulsifiers, thickeners, preservatives, binders, colorants, pigments, solubilizing agents, wetting agents, water-soluble inert fillers, buffering agents, permeation enhancers, and combinations. Thickeners may include gum arabic, carrageenan, karaya gum, gum tragacanth, carob gum, quince seed or Cydonia oblonga, casein, dextrin, gelatin, sodium pectate, sodium alginate, methyl cellulose, ethyl cellulose, CMC, hydroxy ethyl cellulose, hydroxypropyl cellulose, polyvinyl alcohol, PVM, PVP, sodium polyacrylate, carboxy vinyl polymer, locust bean gum, guar gum, tamarind gum, cellulose dialkyl dimethylammonium sulfate, xanthan gum, aluminum magnesium silicate, bentonite, hectorite, AIMg silicate or beagum, laponite, and silicic acid anhydride.

Stabilizing agents may include xanthan gum, locust bean gum and carrageenan, guar gum, sugars, polyols, amino acids or methylamines. Emulsifying agents may include triethanolamine stearate, quaternary ammonium compounds, acacia, gelatin, lecithin, bentonite, sodium benzoate.

When applied to the substrate, particularly when the water-soluble polymer composition is sprayed onto the substrate, the water-soluble polymer composition may have a viscosity in the range of 5,000 mPa-s to 10,000 mPa-s

The water-soluble polymer composition may be formulated such that the freshening active is held in the water-soluble polymer composition until the relative humidity is relatively high in order to delay release of the freshening active until the freshening active is most-needed to combat malodorous compounds. For example, the relative humidity in a trash can may start at about 50% and may increase to at least 70% during normal use. Thus, the selection of the water-soluble-film forming polymer may result in a modulus of the neat water-soluble polymer zone of at least about 2,000 MPa at 40% relative humidity and may reduce in modulus to about 1000 MPa at relative humidity of 75% or greater. When loaded with freshening actives, the modulus may be significantly lower as these can have a plasticizing effect. Here, the modulus of the modified water-soluble polymer zone may be 400 MPa at 40% relative humidity and reduce in modulus to 30 MPa at relative humidity of 75% of greater. Thus, the water-soluble polymer zone holds onto the freshening active until the relative humidity in the trash increases.

The selection of the water-soluble film-forming polymer may improve the wetting and adherence of the water-soluble polymer composition on the substrate. Sufficient wetting and adherence may occur when the water-soluble polymer composition has a contact angle less than 70 degrees.

The polymer should be water soluble/highly swellable such that it will soften when exposed to moisture to release the actives. For example, polymers that only swell in hot water (gelatin) or polymers triggered by acid-base chemistry (chitosan) may be less preferred.

The water-soluble polymer zone may change in opacity or change in color over time as the polymer softens or dissolves in response to moisture from the surrounding environment. The change in opacity or color may be a visual indicator to the consumer that the freshening active is working.

### Freshening Active

The water-soluble polymer composition, when in liquid form, comprises from about 30% to about 80% or from about 40% to about 90%, more preferably from about 40% to about 75% of freshening active.

The freshening active may include one or more substances. The one or more substances may include gaseous, liquid, colloidal suspensions, and/or solid substances. The freshening active may include one or more of volatile fragrance materials (i.e., fragrance materials capable of being transported to the olfactory system) and deodorizing agents (e.g., deodorizing compositions with a deodorizing effect on offensive odors such as that associated with activated nitrogen compound, activated sulfur compounds, etc.). As used herein the term “fragrance” refers to any mixture or composition comprising one or more perfume raw materials with or without one or more carrier solvents configured to emit a pleasant odor. Moreover, as used herein the term “perfume” refers to a compound utilized for its appealing odor. Compounds may have a pleasing odor without being used as a perfume in the context of this disclosure.

Moreover, the freshening active may include one or more of desiccant materials (e.g., a hygroscopic substance, such as calcium oxide or silica gel, that has a high affinity for water and is used as a drying agent), antimicrobial agents (e.g., zinc pyrithione (“ZPT”) and/or copper pyrithione (“CPT”)), deodorizing agents, and functional nanoparticles. The freshening active may include an absorbent agent.

The freshening active may comprise natural oils. For example, the freshening active may include thyme oil, mint oil, lemon grass oil, tea tree oil, cinnamon bark oil, methyl jasmonate, etc.

The freshening active may include a plurality of different components. For example, the freshening active may include a first component of a deodorizing agent and a second component of a volatile fragrance material. In another non-limiting example, the freshening active may include a first component of a deodorizing agent, a second component of an antimicrobial agent, and a third component of a volatile fragrance material. Furthermore, the freshening active may include a plurality of different component to render scents of different expressions (e.g., intensity and/character).

The freshening active may help to reduce an amount of malodorant molecules (e.g., bad smelling molecules) that permeate through or from the substrate of the first and second sidewalls 102, 104 of the bag 100. Additionally, the freshening active may help to control an amount of PRM molecules that permeate through the substrate of the first and second sidewalls 102, 104 of the bag 100. As used herein, the term “permeate” may refer to molecules that pass through the first and second sidewalls 102, 104 or any portions therefore. Furthermore, the term “permeable” and any of its derivative terms when referring to a material means that the material has pores, gaps or other means through which fluids (e.g., gases and/or liquids) can pass. Specifically, when referring to a liquid, no force beyond gravity is necessary for the liquid to move across a liquid-permeable material once that material is saturated with the liquid. When referring to a gas, no force beyond simple diffusion (i.e., the movement of molecules from higher to lower concentrations) is necessary for the gas to move across a gas-permeable material once that material is saturated with that gas.

The freshening active may help prevent malodorant molecules from permeating through the substrate of the bag 100. For example, the freshening active may at least partially absorb and/or trap malodorant molecules that permeate into the freshening active from the interior of the bag 100. In other words, the freshening active may “catch” the malodorant molecules. Air within the freshening active (e.g., air bubbles) may trap the malodorant molecules. Furthermore, the freshening active may trap malodorant molecules by reacting with the malodorant molecules with, for example, reactive substances. The freshening active may also neutralize malodorant molecules by reacting with the malodorant molecules. By absorbing and/or trapping the malodorant molecules, the freshening active may prevent the malodorant molecules from permeating to an exterior of the bag 100. As a result, the bag 100 of the present disclosure may allow less malodorant molecules to permeate through the substrate of the first and second sidewalls 102, 104 of the bag 100 in comparison to sidewalls of conventional thermoplastic bags.

Furthermore, the freshening active may include a plurality of different substances that are configured to be released at different times. For example, the freshening active may include a first odor-control element that releases during a first 24-hr period, a second odor-control element that releases during a second 24-hr period (e.g., hours 24 to 28), and a third odor-control element that releases during a third 24-hr period (e.g., hours 48 to 72). As another non-limiting example, the freshening active may include a first layer that releases a fragrance material initially, a second layer that releases a fragrance material after a certain period of time (e.g., has a delayed release), and a third layer that releases a fragrance material after longer a certain period of time (e.g., has a longer delayed release).

For example, one or more portions of the freshening active may be encapsulated to delay a release of that portion of the freshening active. The one or more portions of the freshening active may be encapsulated within one or more of starch, cyclodextrins starch materials, or perfume microcapsules. The microcapsules may include melamine, polyacrylamide, silicones, silica, polystyrene, polyurea, polyurethanes, polyacrylate based materials, gelatin, styrene malic anhydride, polyamides, and mixtures thereof. Additionally, the microcapsules may include melamine crosslinked with formaldehyde, melaminedimethoxyethanol crosslinked with formaldehyde, and mixtures thereof. The microcapsules may include polystyrene cross-linked with divinylbenzene, urea crosslinked with formaldehyde, urea crosslinked with gluteraldehyde, polyacrylate formed from methylmethacrylate or dimethylaminomethyl methacrylate, polyacrylate formed from amine acrylate and/or methacrylate and strong acid, polyacrylate formed from carboxylic acid acrylate and/or methacrylate monomer and strong base, polyacrylate formed from an amine acrylate and/or methacrylate monomer and a carboxylic acid acrylate and/or carboxylic acid methacrylate monomer, and mixtures thereof. Furthermore, the perfume microcapsule may be coated with a deposition aid, a cationic polymer, a non-ionic polymer, an anionic polymer, or mixtures thereof. Suitable polymers may include polyvinylformaldehyde, partially hydroxylated polyvinylformaldehyde, polyvinylamine, polyethyleneimine, ethoxylated polyethyleneimine, polyvinylalcohol, polyacrylates, and combinations thereof. The freshening active may include perfume material complexes (e.g., materials used in Schiff base reactions). In other words, the freshening active may include catalysts used to at least partially neutralize malodorant molecules. The shell of encapsulated freshening actives may be biodegradable.

The freshening active may produce malodor reduction without an added fragrance. Such a freshening active can be used to provide an unscented bag or used in combination with a fragrance.

The water-soluble polymer composition may be able to control the release or exposure of the freshening actives, allowing for the use of freshening actives that would otherwise be avoided on substrates because the freshening active may be considered a skin irritant or may be associated with inhalation concerns under some use conditions. For example, the freshening active may comprise calcium carbonate, magnesium carbonate, barium carbonate, alumina, magnesium oxide, zinc oxide, superabsorbent polymers, calcium chloride, zeolite (aluminosilicates), pulp (wood) powder, or any combination thereof. The freshening active may comprise hydrogen peroxide, peroxydone, halohydantoins, magnesium hydroxide hypochlorite oxide, sodium perborate, sodium percarbonate, or acid catalysts.

Likewise, use of the water-soluble polymer composition may allow for the inclusion of freshening active that would otherwise be avoided on substrates because the freshening actives are wet or sticky. As used herein, the term “sticky” may refer to a material that tending to stick to (e.g., at least partially attach to) surfaces upon contact.

For example, the freshening active can comprise copper chloride colloidal nanoparticles, stabilized oxidants such as an intercalated bleach, or metal salts of polyitaconic acid resins (i.e., poly (sodium zinc itaconate). For example, the freshening active can comprise polyethylene glycol copolymers, polyethylenimine, or silicone.

The intercalated bleach compounds that may be used as freshening actives in the present invention include an alkaline earth hypochlorite species intercalated with oxide and/or hydroxide species. The intercalated bleach compounds exhibit excellent stability (e.g., equal to or better than any other known chlorine bleach species), little or no characteristic chlorine bleach odor as compared to other forms of chlorine bleach, exhibit excellent pH buffering characteristics at significantly gentler pH ranges (e.g., about 8 to about 11.5) than existing liquid bleach compositions (11.5 to 13.5). The intercalated bleach compound is stable, even in high humidity environments, and shows relatively less reactivity with organic materials as compared to other solid chlorine bleach alternatives. The material does not appear to show evidence of any self-propagating decomposition reactions, can be provided in solid form (which can be dissolved or suspended into aqueous solution), and does not readily clump or cake as do many existing alternative chlorine bleaches. The material exhibits better flexibility as to its compatibility with various adjuvants than existing alternatives, can be formulated to control release of hypochlorite over a desired period of time, and may be formulated within compositions that are phosphate free while providing the above benefits.

The available chlorine concentration in the intercalated bleaching compound may be from about 0.01% to about 25%, or from about 0.1% to about 25% %, or from about 1% to about 25%, or from about 2.5% to 25%.

The intercalated bleach compound may have the formula Mₓ(OCl)_{y}(O)ₘ(OH)ₙ:
wherein M is an alkaline earth metal or mixture of alkaline earth metals, such as magnesium or calcium or mixtures thereof;
wherein x and y independently equal any number greater than or equal to 1 (e.g., 1, 2, 3, 4, etc.);
wherein m and n independently equal 0 or any number greater than 0 (e.g., 0, 1, 2, 3, 4, etc.), but m and n are not both 0; and
wherein x is ≥3y.

The values of x, y, m, and n may be integers (i.e., whole numbers). By way of further example, in an embodiment, 2m+n=5y. In another embodiment, x=0.5y+m+0.5n.

### Bag

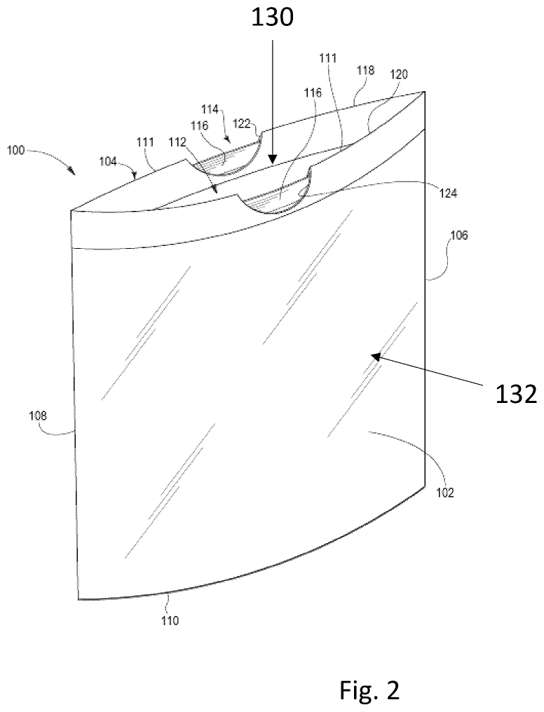

FIG. 2 is a perspective view of an exemplary, non-limiting example of a thermoplastic bag 100. The bag 100 includes a first sidewall 102 and a second sidewall 104. Each of the first and second sidewalls 102, 104 includes a first side edge 106, a second opposite side edge 108, a bottom edge 110 extending between the first and second side edges 106, 108, and top edge 111 extending between the first and second side edges 106, 108 opposite the bottom edge. The first sidewall 102 and the second sidewall 104 are joined together along the first side edges 106, the second opposite side edges 108, and the bottom edges 110. The first and second sidewalls 102, 104 may be joined along the first and second side edges 106, 108 and bottom edges 110 by any suitable process such as, for example, a heat seal. The first and second sidewalls 102, 104 may not be joined along side edges. Rather, the first and second sidewalls 102, 104 may be a single uniform piece. In other words, the first and second sidewalls 102, 104 may form a sleeve or a balloon structure, defining an interior 130 and an exterior 132.

The bottom edge 110 or one or more of the side edges 106, 108 can comprise a fold. In other words, the first and second sidewalls 102, 104 may comprise a single unitary piece of material. The top edges 111 of the first and second sidewalls 102, 104 may define an opening 112 to an interior of the bag 100. In other words, the opening 112 may be oriented opposite the bottom edge 110 of the bag 100. Furthermore, when placed in a trash receptacle, the top edges 111 of the first and second sidewalls 102, 104 may be folded over the rim of the receptacle.

The bag 100 may optionally include a closure mechanism 114 located adjacent to the top edges 111 for sealing the top of the bag 100 to form an at least substantially fully-enclosed container or vessel. As shown in FIG. 2, the closure mechanism 114 may comprise a draw tape 116, a first hem 118, and a second hem 120. In particular, the first top edge 111 of the first sidewall 102 may be folded back into the interior volume and may be attached to an interior surface of the first sidewall 102 to form the first hem 118. Similarly, the second top edge 111 of the second sidewall 104 is folded back into the interior volume and may be attached to an interior surface of the second sidewall 104 to form a second hem 120. The draw tape 116 extends through the first and second hems 118, 120 along the first and second top edges 111. The first hem 118 includes a first aperture 122 (e.g., notch) extending through the first hem 118 and exposing a portion of the draw tape 116. Similarly, the second hem 120 includes a second aperture 124 extending through the second hem 120 and exposing another portion of the draw tape 116. During use, pulling the draw tape 116 through the first and second apertures 122, 124 will cause the first and second top edge 110 to constrict. As a result, pulling the draw tape 116 through the first and second apertures 122, 124 will cause the opening 112 of the bag to at least partially close or reduce in size. The draw tape closure mechanism 114 may be used with any of the implementations of a reinforced thermoplastic bag described herein.

Although the bag 100 is described herein as including a draw tape closure mechanism 114, one of ordinary skill in the art will readily recognize that other closure mechanisms 114 may be implemented into the bag 100. For example, the closure mechanism 114 may include one or more of flaps, adhesive tapes, a tuck and fold closure, an interlocking closure, a slider closure, a zipper closure, or any other closure structures known to those skilled in the art for closing a bag.

FIG. 3 is a side cross-sectional view of the bag 100 of FIG. 2. The bag 100 comprises one or more water-soluble polymer zones 12 on the interior 130 surface of one or more sidewalls 102, 104 of the bag 100.

The bag 100 of the present invention may maintain a fresher (e.g., cleaner) smell when compared to conventional thermoplastic bags even when contents of the bag 100 have a bad odor. As a result, rooms in which the bags 100 are used as trash bags may maintain a better smell. Furthermore, the bag 100 of the present disclosure may reduce an amount of malodorant molecules that come into contact with a receptacle (e.g., trash can) in which the bag 100 may be inserted. Accordingly, receptacles using bags 100 of the present disclosure may smell better than receptacles using conventional thermoplastic bags. As an additional result, receptacles using bags 100 of the present disclosure may require less cleaning than receptacles using conventional thermoplastic bags.

As noted briefly above, additional trigger mechanisms, in addition to or separate from moisture, for activating the freshening active may include activating the freshening active via pressure and/or friction on the substrate caused by articles placed in bag 100. In other words, the freshening active may be “touch activated.” As also mentioned above, the trigger mechanisms for activating the freshening active may further include thermal activation (e.g., heat activating), chemical activation (e.g., using internal freshening active chemistry to cause a reaction), photolytic activation (e.g., using light to activate freshening active), and/or pH activation (e.g., using pH value to activate freshening active).

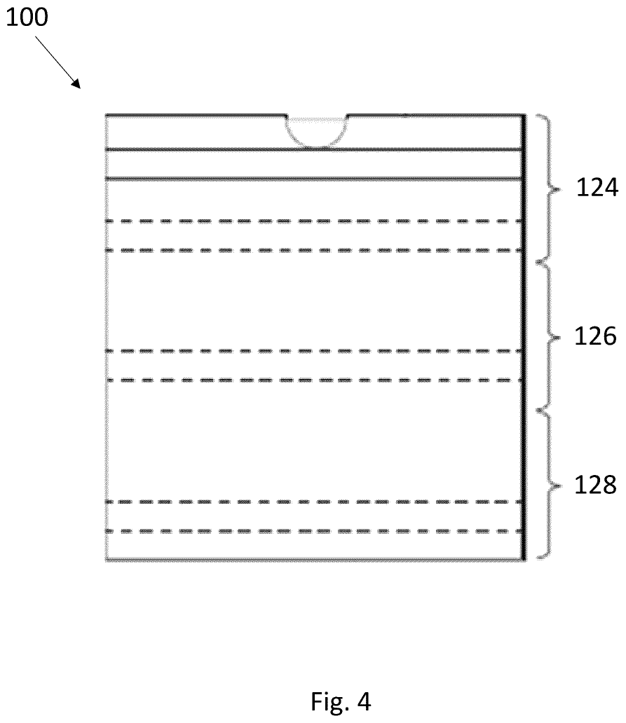

FIG. 4 shows a plan view of an exterior sidewall of a thermoplastic bag 100, illustrating freshening active placement regions of the thermoplastic bag 100. In particular, the water-soluble polymer zone may be disposed in one or more regions on the interior surfaces of the sidewalls of the thermoplastic bag. The water-soluble polymer zones may be disposed in a middle region 126 of the thermoplastic bag 100. The freshening active may be disposed in a lower region 128 of the thermoplastic bag 100. For example, the freshening active may be disposed at a bottom of the thermoplastic bag 100. The freshening active may be disposed in two or more of the upper, middle, and lower regions 124, 126, 128 of the thermoplastic bag 100.

In a non-limiting example, freshening actives that require friction to release the freshening active may be disposed in the upper region 124 so as to release as the bag is filled and or compacted by a user. In another non-limiting example, a freshening active that requires dissolution of the water-soluble polymer composition to release the freshening active may be disposed in the middle or lower regions 126 or 128 where the humidity or liquid content may be relatively higher than other areas of the bag. Some substrates may include water-soluble polymer zones with freshening actives at one or more regions that include the same or different freshening actives.

The water-soluble polymer zones may take one or more different shapes. For example, the water-soluble polymer zones may have a rectangle shape, a square shape, a circle shape, a triangle shape, or any other geometric shape, and combinations thereof. The water-soluble polymer zones may have a random droplet pattern, as the result of being sprayed or printed onto the substrate. The water-soluble polymer zones may be discontinuously dispersed on the substrate in regular or irregular patterns.

The water-soluble polymer zones may be relatively small in size in order to provide a strong bond between the water-soluble polymer zones and the substrate. The water-soluble polymer zones may each have a surface area of 10 to 300 cm².

The water-soluble polymer zones may be various colors and may provide an additional aesthetic benefit to the bag. The water-soluble polymer zones may be similar to the color of the bag in order to be less visible to a consumer or may be a different color than the bag in order to increase visibility of the water-soluble polymer zones to the consumer.

### Method of Making Substrates Comprising Water-Soluble Polymer Zones

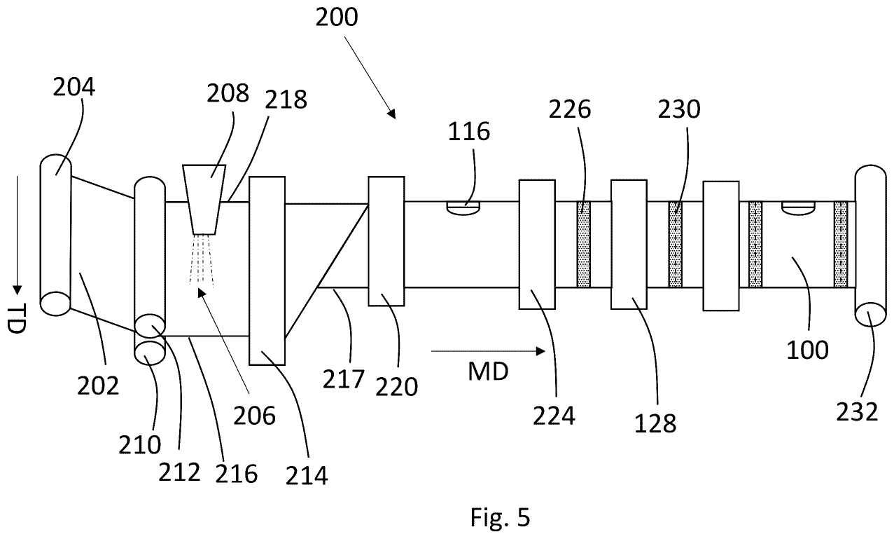

FIG. 5 illustrates a manufacturing process for making substrates having water-soluble polymer zones disposed thereon and then producing plastic bags therefrom. FIG. 5 illustrates an exemplary consumer product method of manufacture for making bags. This method is illustrative of the methods of manufacture for making various consumer products according to the present invention. Referring to FIGS. 1, 2, and 5, according to the process 200, one or more substrates in the form of films 202, for example, may be unwound from stock rolls 204, respectively, and directed along a machine direction MD. Alternatively, the film may be directly from one or more extrusion towers rather than stock rolls 204.

The water-soluble polymer composition 206 (i.e., one or more substances) may be applied to one or more of the substrates using an applicator 208. The application may include spraying, printing, and any other method known in the art for applying liquid substances to substrates. Once applied to the substrates, the water-soluble polymer composition 206 is allowed to dry. The water-soluble polymer composition may be air-dried, or dried with assistance of air, heat, light. Preferably, the water-soluble polymer composition is capable of being air-dried onto the substrates to form a substantially dry, water-soluble polymer zone that dries in a relatively short time so that the substrates can be further processed on the manufacturing line. For example, the water-soluble polymer zone may preferably dry in less than 2 minutes, more preferably less than 1 minute. More preferably, the water-soluble polymer composition forms a non-sticky, water-soluble polymer zone on the substrates that can be folded to form bags, for example, without the water-soluble polymer zone sticking to other portions of the substrates.

After the water-soluble polymer composition has been applied to the film, the film may be passed between a pair of cylindrical intermeshing rollers 210, 212 to incrementally stretch. The intermeshing rollers 210, 212 shown in FIG. 5 may have a construction similar to that of any of the intermeshing rollers described in U.S. Pat. No. 8,603,609. The rollers 210, 212 may be oriented such that longitudinal axes of the rollers are perpendicular to the machine direction. Additionally, the rollers 210, 212 may rotate about their longitudinal axes in opposite rotational directions. Motors may be provided to power rotation of the rollers 210, 212 in a controlled manner. As the film passes between the pair of rollers 210, 212, the ridges and/or teeth of the rollers 210, 212 can form the film (i.e., eventual sidewall of the bag 100).

A folding operation 214 can fold the film to produce the sidewalls of the finished bag. The folding operation 214 can fold the film in half along the transverse direction. In particular, the folding operation 214 can move a first edge 216 adjacent to the second edge 218, thereby creating a folded edge 217. For example, the process may include the folding operation described in U.S. Pat. No. 8,568,283, the entire contents of which are hereby incorporated by reference in their entirety. Additionally, the folding operation 214 may form a hem at an eventual top portion of a thermoplastic film.

To produce the finished bag, the processing equipment may further process the folded film. In particular, a draw tape operation 220 can insert a draw tape 116 into edges 216, 218 of the film. Furthermore, a sealing operation 224 can form the parallel side edges of the finished bag by forming heat seals 226 between adjacent portions of the folded film. Moreover, the sealing operation 224 can seal the hem to a sidewall of the eventual thermoplastic bag. The heat seal 226 may strongly bond adjacent layers together in the location of the heat seal 226 so as to tightly seal the edges (e.g., produce an at least substantially water tight seal) of the finished bag. The heat seals 226 may be spaced apart along the folded film to provide a desired width to the finished bags. The sealing operation 224 can form the heat seals 226 using a heating device, such as, a heated knife or seal bar.

A perforating operation 228 may form a perforation 230 in the heat seals 226 using a perforating device, such as, a perforating knife. The perforations 230 in conjunction with the folded outer edge 220 can define individual bags 100 that may be separated from the film. A roll 232 can wind the film embodying the finished bags 100 for packaging and distribution. For example, the roll 232 may be placed into a box or bag for sale to a customer.

In still further implementations, the folded film may be cut into individual bags along the heat seals 226 by a cutting operation. In another implementation, the folded film may be folded one or more times prior to the cutting operation. In yet another implementation, the side sealing operation 224 may be combined with the cutting and/or perforation operations 228.

### Test Methods

### Contact Angle of Water-Soluble Polymer Composition Method

A glass slide was covered with a cut section from the non-patterned area of a Glad Forceflex trash bag. 3 μL of solution (90% of malodor ingredient, 20% active and 10% soluble film, 20-50% active) was pipetted onto the film. An image was taken with a Celestron Microscope+Microcapture Pro. Images were then analyzed by the measure angle feature of ImageJ. Three drops/specimens of each polymer composition were analyzed. Sample images are shown below.

### Drying Time

Drying time was calculated as the amount of time from when deposition was complete to when water-soluble polymer zone were dry visually and did not leave any residue when tapped with a finger.

### Adhesion

Adhesion of the water-soluble polymer composition was assessed approximately 24 hours after the water-soluble polymer zones were cast onto the bags. Bags were fluffed open to simulate a consumer experience, and if any flaking was observed it was noted. Additionally, the water-soluble polymer zones were touched gently to see if the films were tacky to the touch or if any water-soluble polymer zones rubbed off.

### Modulus of Water-Soluble Polymer Composition

Tensile (modulus) properties are measured on a constant rate of extension tensile tester with computer interface (a suitable instrument is the MTS Insight using Testworks 4.0 Software, as available from MTS Systems Corp., Eden Prairie, MN) using a load cell for which the forces measured are within 1% to 99% of the limit of the cell. Both the movable (upper) and stationary (lower) fixture are fitted with light weight vise action grips, wider than the width of the test specimen. The tensile tester is fitted with a specimen humidity and temperature control chamber that can maintain the specimen's environment at a set temperature between 23° C. and 40° C. with an accuracy of ±0.5° C. and at a set relative humidity of between 35% and 95% with an accuracy of ±0.5% throughout the tensile experiment. All testing is performed in a conditioned room maintained at about 23° C.±2° C. and about 50%±2% relative humidity.

Program the tensile tester for an extension test after performing an initial gage length adjustment. To perform the gage-length adjustment, first lower the crosshead 5.0 mm at a rate of at 7.5 mm/s to add slack to the specimen. Then raise the crosshead at 7.5 mm/s until 0.1 N is measured at the load cell, and set the current gage at this point as the adjusted gage length. Continue to raise the crosshead at 7.5 mm/s until the specimen breaks, i.e., that the force drops to <0.05 N after the maximum peak force. Force and crosshead travel data are collected at 200 Hz throughout the experiment. Return the crosshead to the original gage length. Samples are conditioned at about 23° C.±2° C. and about 50%±2% relative humidity for at least two hours before testing. Determine the machine direction (MD) and cross direction (CD) of the samples. Using a JDC cutter (available from Thwing-Albert) or other appropriate means, cut eighteen (18) specimens 80 mm long by 25.4±0.1 mm wide in the cross direction. Next cut eighteen (18) specimens 80 mm long by 25.4±0.1 mm wide in the machine direction. Measure the caliper of each specimen using a digital linear caliper (e.g. an Ono Sokki GS-503 or equivalent) fitted with a 25 mm diameter foot that applies a pressure of 0.69 kPa. Zero the caliper foot against the anvil base. Lift the foot and place the specimen flat against the anvil base, with the specimen width centered beneath the pressure foot, and lower the foot at about 5 mm/sec onto the specimen avoiding any creases, folds, or obvious defects. Read the caliper (mm) 5.0 sec after resting the foot on the specimen and record to the nearest 0.01 mm. Calculate the cross sectional area of each specimen as the width of the specimen multiplied by the caliper, and record to 0.01 mm².

Accurately set the initial gage length to 75.0 mm and zero the crosshead and load cell. Insert the specimen into the upper grips, aligning it vertically within the upper and lower grips, close the upper grip and tighten. Insert the opposite end of the specimen into the lower grip and tighten. The specimen should have minimal slack with less than 0.1 N force measured at the load cell. Seal the environmental chamber and allow the temperature and humidity to reach the set target values, and then maintain these conditions for 10 minutes prior to testing. Start the test program.

Plot the Force (N) versus Extension (mm) curve. Herein, Extension is the travel length corrected for the adjusted gage length. Read and record the maximum Peak Force and report to the nearest 0.1 N. Calculate the Energy to Break as the area under the curve between the start of extension to the final extension at break. Record to the nearest 0.01 N*mm.

Using the force (N) and extension data (mm), construct an engineering Stress (MPa) versus engineering Strain curve. Herein engineering stress s is defined as force (N) divided by the initial cross sectional area (mm²) of the specimen. Engineering strain e is the change in length (from the adjusted gage length) divided by the adjusted gage length. From the curve read the Failure Stress (MPa) as the maximum stress of the curve and record to the nearest 0.01 MPa. From the curve, calculate the Modulus (MPa) as the greatest slope of a linear segment fitted by linear regression to the curve, wherein the length of the segment incorporates at least 20% of the curve. Record to the nearest 0.01 MPa.

The analysis is performed for three replicate CD and three replicate MD specimens, at each of the following target environmental conditions:

| | Temperature (° C.) | Relative Humidity (%) |
|---|---|---|
| 1 | 25 | 40 |
| 2 | 25 | 75 |
| 3 | 25 | 90 |

Calculate the arithmetic mean for the three replicate CD results at each environmental condition. Separately calculate the arithmetic mean for the three replicate MD results at each environmental condition, report Modulus to the nearest 0.1 MPa, Failure Stress to the nearest 0.1 MPa, Peak Force to the nearest 0.01 N and Energy to Break to the nearest 0.1 N*mm for each of the environmental conditions.

### Sensory Analysis of Fragrance and Malodors

Discrimination testing requires screened and knowledgeable respondents. This trained group has the ability to measure and provide input on a variety of trash needs including product reformulation, alternate supplier approval, or other discernment needs. Good sensory practices are followed when preparing the test environment, the substrate and in training the panel.

Synthetic and actual malodors are used. When actual malodors are used, care is taken to ensure sample uniformity, limiting variability in odor profiles from test to test.

Measurement of the test product performance is done using both (1) an untreated malodor control and (2) a combination of the specified malodor and the product. Testing occurs in isolated chambers and after the appropriate exposure time for the malodorant, product, or both has elapsed, evaluation of each material occurs while that material remains in its isolated chamber. Care is taken to ensure an odorless state within the evaluation space.

Trash bags comprising water-soluble polymer zones are then placed into standard 13-gallon trash cans (standard household trash cans with lid). Cans are then loaded with the malodor being tested. Additionally, the untreated malodor control (a trash bag with no added perfume accords) is loaded with the malodor being tested.

Depending on the test objective, either a specific malodor component or a proprietary trash mix representative of consumer household waste is added to the trash bags (both treated and control), covered and allowed to age for the appropriate time point.

Samples are presented to assessors in odor evaluation chambers that are labeled with randomly generated codes. Assessors are asked to smell the chamber contents and evaluate the overall intensity. Assessors are asked to rest between evaluations. Using a 15-point universal scale, the panel evaluates each sample by Total Intensity=Total Malodor+Total Fragrance. Data are statistically analyzed using ANOVA with pairwise comparisons analyzed using Fischer's LSD and interpreted at the 95% confidence level. Data are statistically analyzed using a One-Way ANOVA with Fischer's LSD.

### Shear Methodology

Encapsulation samples are evaluated both Pre-Activated and Post Activated following general panel assessment guidelines. Each panelist evaluates a non-activated sample for Total Intensity, Total Malodor and Total Fragrance. After all panelists have completed the Pre-Activation assessment, a proprietary activation device is used on each sample to shear the walls of the trash bag. The panel then completes a Post-Activated assessment in the same manner at appropriate time points.

### EXAMPLES

The following are non-limiting examples of products and methods of using the present invention. The examples are given solely for the purpose of illustration and are not to be construed as limitations of the present invention, as many variations thereof are possible without departing from the spirit and scope of the invention, which would be recognized by one of ordinary skill in the art.

In the examples, all concentrations are listed as weight percent, unless otherwise specified and may exclude minor materials such as diluents, filler, and so forth. The listed formulations, therefore, comprise the listed components and any min or materials associated with such components. As is apparent to one of ordinary skill in the art, the selection of these minors will vary depending on the physical and chemical characteristics of the particular ingredients selected to make the present invention as described herein.

### Example 1: Evaluating Contact Angle of Water-Soluble Polymer Compositions

### Water-Soluble Polymer Concentrate Preparation Methods

To create the freshening formulas, first aqueous concentrates of the polymers were created. These were either tested as is or were combined with freshening actives for additional testing. Two examples of polymer concentrates are given below:

### METHOCEL™ Concentrate Preparation Method

Solutions of the METHOCEL™ polymers were made as follows. First, distilled water is added to a beaker and heated to 95° C. with stirring from an overhead mixer at 300 RPM. Once at 95° C., 1% of phenoxyethanol is added and stirred until dispersed. Next, 20% METHOCEL™ is added with stirring at 300 RPMs. Once all the polymer is added, the heat is removed until the solution reaches 60° C. Once at 60° C., the stirring is reduced to 150 RPMs and the solution is placed in an ice bath until completely hydrated.

### Polyethylene Oxide Concentrate Preparation Method

Solutions of the polyethylene oxide (PEO) polymers were made as follows. First, distilled water is added to a beaker and stirred with an overhead mixer at 500 RPM. Then, 1% phenoxyethanol is added. Once the phenoxyethanol is dispersed, 50% PEO is slowly added over 45 minutes. Stirring is continued for an additional 30 minutes and then the stirring rate is reduced to 200 RPM. The mixture is then stirred for an additional 2 hours.

Other polymers not described in detail were added in a similar manner to create aqueous concentrates ranging from 11.5%-50% polymer as noted in Table 1.

### Freshening Active Additions (Aqueous):

Most aqueous and/or water-soluble freshening actives can be added directly to the polymer concentrates at room temperature with stirring. The freshening compositions are added at levels ranging from 80%-90% by weight.

The contact angle for Example Formulas A through F are provided in Table 1. Without wishing to be bound by theory, the lower the contact angle, the greater the spreading of the water-soluble polymer composition and the greater the adherence to the film.

TABLE 1

| Example Formula | A | B | C | D | E | F |
|---|---|---|---|---|---|---|
| Water-Soluble, Film- | — | METHOCEL ™ | PVP | PEG8000 | — | METHOCEL ™ |
| Forming Polymer | | | | | | |
| Water-Soluble, Film- | — | 2% | 5% | 5% | — | 4% |
| Forming Polymer (%) | | | | | | |
| Water | Balance | Balance | Balance | Balance | Balance | Balance |
| Freshening active A (%) | 100% | 90% | 90% | 90% | — | — |
| Freshening active B (%) | — | — | — | — | 100% | 80% |
| Contact Angle (Degrees) | 88 | 63 | 87 | 87 | 84 | 61 |

### Example 2: Evaluating Drying Time and Adhesion of Water-Soluble Polymer Compositions

### Preparation of Films Comprising Water-Soluble Polymer Zones:

Polymer concentrates were prepared as described in Water-soluble Polymer Concentrate Preparation Methods. After concentrates had been made, the example water-soluble polymer concentrates were deposited via screen printing onto the film. A screen of mesh size 420 and a blocking stencil with a net pattern was used as follows: The screen is placed on top of a Mylar film to be used as a proxy substrate. A few milliliters of sample are placed on the mesh and a squeegee is used to force the solution through the mesh and onto the substrate. The mesh is then gently removed. The samples which transfer to the Mylar substrates are then observed to determine if they dry and how well they adhere to the Mylar substrate.

TABLE 2

| Polymer Name | Drying Time | Wt. % Polymer | Adhesion to Plastic Film |
|---|---|---|---|
| METHOCEL ™ E3LV | 50 sec | 20% | Yes |
| Polyethylene Glycol | >120 sec | 50% | Flakes off of film |
| 4000 | | | |
| Polyethylene Glycol | >120 sec | 50% | Flakes off of film |
| 8000 | | | |
| PVP K30 | Never dried | 50% | Yes (tacky film) |
| FLORASOLVS ™ | Never dried | 20% | No |
| (PEG-100 Hydrogenated | | | |
| Jojoba) | | | |
| METHOCEL ™ E5LV + | Never dried | 10% METHOCEL ™ | No |
| FLORASOLVS ™ Wax | | 10% FLORASOLVS ™ | |
| METHOCEL ™ | Never dried | 10% METHOCEL ™ | No adhesion. De- |
| E5LV + PEG4000 | | 25% PEG 4000 | wetting/beads out. |
| METHOCEL ™ | Never dried | 10% METHOCEL ™ | No adhesion. De- |
| E5LV + PEG8000 | | 25% PEG 8000 | wetting/beads out. |
| Alginate | Never dried | 12% | No |

### Example 3: Evaluation of Modulus of Water-Soluble Polymer Zones

### Formula Preparation

Polymer concentrates for the moduli studies were prepared as described previously in the section Water-soluble Polymer Concentrate Preparation Methods.

To create water-soluble polymer zones containing glycerin, the following method was used. After the polymer concentrates were prepared, 10.52% by weight of glycerin is added to the polymer concentrate in a beaker at room temperature. These ingredients are then mixed together until homogenous.

### Water-Soluble Polymer Composition Casting

Water-soluble polymer zones are created by solution casting with a Bird drawdown bar to create water-soluble polymer zones of uniform thickness.

TABLE 3

| | | Dried | | | |
|---|---|---|---|---|---|
| Water-Soluble | Wet | Concentration | Modulus (MPa) | | |
| Polymer Composition | Concentration | (approximate) | 40% RH | 75% RH | 90% RH |
| METHOCEL ™ | 20% | 100% | 1925 | 1122 | 61 |
| | METHOCEL ™ | METHOCEL ™ | | | |
| Polyethylene Oxide | 20% PEO | 100% PEO | 395 | 224 | 38 |
| 200K | | | | | |
| METHOCEL ™ | 18% METHOCEL ™ | 63% METHOCEL ™ | 417 | 30 | 5 |
| Glycerin | 10.5% Glycerin | 37% Glycerin | | | |
| Polyethylene Oxide | 18% PEO | 63% PEO | 126 | 23 | — |
| 200K Glycerin | 10.5% Glycerin | 37% Glycerin | | | |

### Results

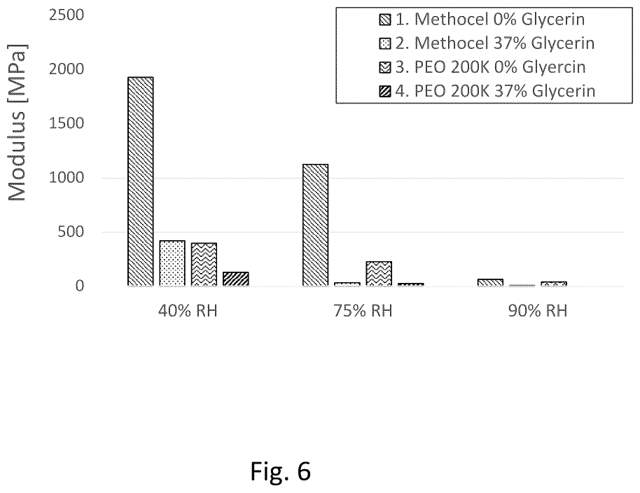

With reference to Table 3 and FIG. 6, these data demonstrate that the mechanical properties of these water-soluble polymer zones are sensitive to relative humidity. At low relative humidity, such as would be found under typical storage conditions, the water-soluble polymer zones should have high modulus and act to contain any active ingredients. However, as the relative humidity increases, such as might be found in-use conditions in a trash receptable with food waste, the modulus will decrease enabling active ingredients to be released from the water-soluble polymer zones.

These data further show that the overall modulus and the sensitivity of the modulus to relative humidity can be targeted with specific ingredients. Glycerin, in this case, was successfully employed to sharply decrease the modulus and enhance the response to relative humidity. It is expected that several of the freshening actives will have similar effects and can serve a dual purpose of a freshening benefit and helping to control the modulus effect.

### Example 4: Evaluation of Fragrance Intensity Over Time

### Freshening Active Addition (Non-Aqueous):

Lipophilic ingredients are not readily miscible in the polymer concentrates. For lipophilic freshening actives, the following methodology was employed: To the polymer concentrate, 0.08% of polysorbate 20 is added. Then, the freshening active(s) are added at 40% by weight. Once the actives and the surfactant are added, the mixture is milled with an IKA T25 Turrax homogenizer for 3-5 minutes at 4000 rpm to emulsify the system.

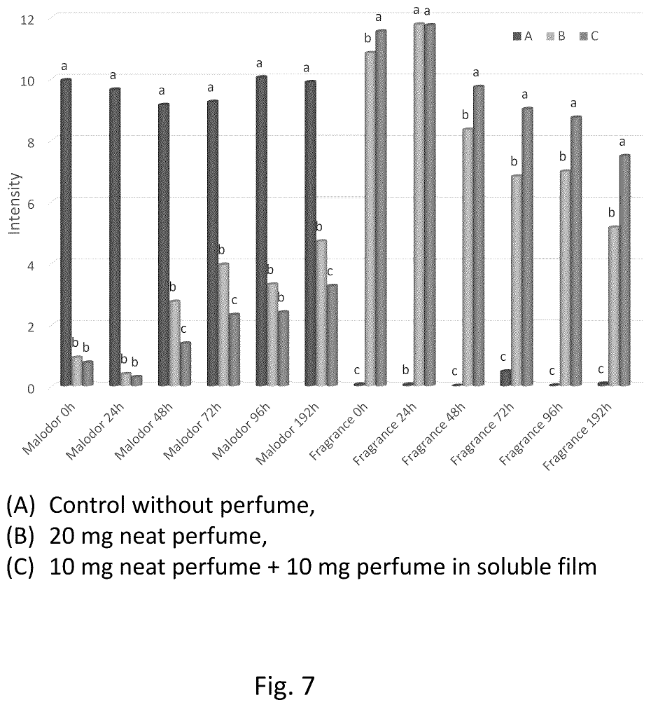

The resulting water-soluble polymer compositions were evaluated for fragrance and malodor intensity according to the Sensory Analysis of Fragrance and Malodors test method described above. The results are illustrated in FIG. 7.

### Example 5: Evaluation of Encapsulated Freshening Actives

Bags comprising water-soluble polymer compositions with encapsulated freshening actives were made as follows: First, METHOCEL™ concentrates were made according to the METHOCEL™ Concentrate Preparation Method described above. The METHOCEL™ Concentrate is added to the encapsulate slurry at 20 weight % with gentle stirring at room temperature in an appropriate mixing vessel. The appropriate amount of the encapsulate-METHOCEL™ mixture is then dosed onto a 12″ section on the inside of the bag. The bag is then closed and a 5-lb hand-roller is rolled across the outside of the region where the formula was dosed one time to generate a consistent water-soluble polymer zone.

The resulting water-soluble polymer compositions were evaluated for scent intensity according to the Sensory Analysis of Fragrance and Malodors test method described above. The results are illustrated in FIG. 8.

### Example 6: Evaluation of Intercalated Bleach Freshening Actives

Bags comprising water-soluble polymer compositions with intercalated bleach freshening actives were made as follows: First, METHOCEL™ concentrates were made according to the METHOCEL™ Concentrate Preparation Method described above. Bleach freshening actives were added to the METHOCEL™ concentrates at 15 weight % with stirring. Compositions were then applied to the inside of the bag with typical fragrance dosing procedures.

Bags comprising intercalated bleach freshening actives added between layers instead of inside the bag were made as follows: Bleach freshening actives with mixed with propylene glycol at 40 weight %. Compositions were applied between the bag layers with typical fragrance dosing procedures.

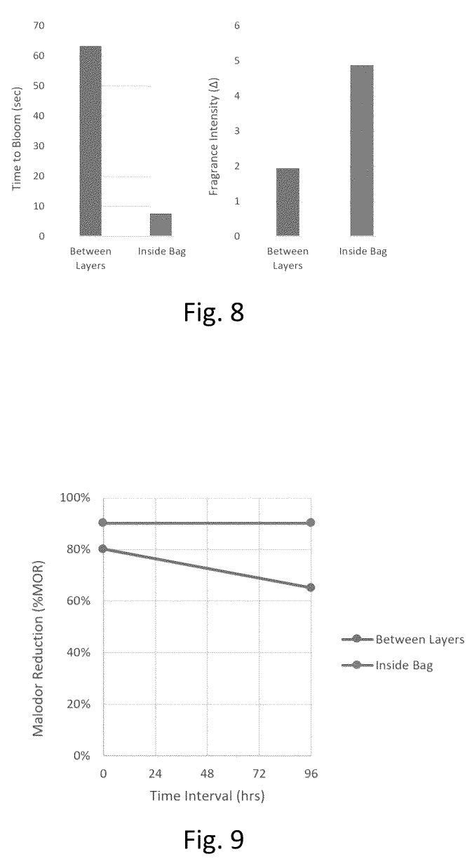

The resulting water-soluble polymer compositions were evaluated for scent intensity according to the Sensory Analysis of Fragrance and Malodors test method described above. The results are illustrated in FIG. 9.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1-20. (canceled)

21. A consumer product comprising:
a polyethylene film comprising a surface;
a plurality of water-soluble polymer zones disposed on and in contact with the surface, the plurality water-soluble polymer zones comprising a freshening active and a polymer,
wherein the consumer product is free of an adhesive that is disposed between the surface of the polyethylene film and the water-soluble polymer zone.

22. The consumer product of claim 21, wherein the freshening active is selected from the group consisting of: a perfume raw material, malodor counteractant, and combinations thereof.

23. The consumer product of claim 21, wherein the freshening active is encapsulated.

24. The consumer product of claim 21, wherein the polymer is selected from the group consisting of: methyl cellulose, hydroxyethyl methyl cellulose, hydroxypropyl methyl cellulose, hydroxypropyl ethyl cellulose, ethyl cellulose, hydroxyethyl methyl cellulose, and combinations thereof.

25. The consumer product of claim 21, wherein the water-soluble polymer zone comprises at least 70 wt. % of a freshening active.

26. The thermoplastic film of claim 21 wherein the plurality of water-soluble polymer zones are discontinuously dispersed.

27. The consumer product comprising of claim 21, wherein the consumer product is selected from the group consisting of: wipe, duster, dry mop pad, air filter, dryer sheet, bag, film, diaper, sanitary napkins, incontinence products, fiber-based detergent, paper towel, toilet paper, foam sponge, and combinations thereof.

28. A method of making a consumer product, the method comprising the steps of:
providing a substrate;
depositing a liquid, water-soluble polymer composition onto one or more locations of the substrate, wherein the liquid, water-soluble polymer composition comprising a freshening active, a polymer, and a surfactant; and
drying the liquid, water-soluble polymer composition on the surface of the substrate to form one or more water-soluble polymer zones.

29. The method of claim 28, wherein the freshening active is selected from the group consisting of: a perfume raw material, malodor counteractant, an intercalated bleach, cleaning active, antibacterial active, and combinations thereof.

30. The method of claim 29, wherein the freshening active is encapsulated.

31. The method of claim 29 wherein the polymer is selected from the group consisting of: methyl cellulose, hydroxyethyl methyl cellulose, hydroxypropyl methyl cellulose, hydroxypropyl ethyl cellulose, ethyl cellulose, hydroxyethyl methyl cellulose, and combinations thereof.

32. The method of claim 29, wherein the surfactant is selected from the group consisting of: ethoxylated sorbitan, sorbitan, and combinations thereof.

33. The method of claim 29, wherein the water-soluble polymer zone comprises at least 70 wt. % of a freshening active.

34. The method of claim 29, wherein the step of depositing a liquid, water-soluble polymer composition onto one or more locations of the substrate further comprises depositing the liquid, water-soluble polymer composition onto multiple locations of the substrate, and wherein the step of drying the liquid, water-soluble polymer composition on the surface of the substrate to form one or more water-soluble polymer zones further comprises drying the liquid, water-soluble polymer composition on the surface of the substrate to form multiple, discontinuously dispersed water-soluble polymer zones.

35. The method of claim 29, wherein the water-soluble polymer composition has a contact angle less than 70 degrees on the thermoplastic film.

36. The consumer product of claim 29, wherein the modulus of the water-soluble polymer zone may be at least 400 MPa at 40% relative humidity and reduce in modulus to 30 MPa or less at relative humidity of 75% of greater.
